(19) United States
(12) Patent Application Publication
(10) Pub. No.: US 2003/0036700 A1
(43) Pub. Date: Feb. 20, 2003

(54) Internal/external coincident gamma camera system

(76) Inventor: Irving N. Weinberg, Bethesda, MD (US)

Correspondence Address: PATENT ADMINSTRATOR, KATTEN MUCHIN ZAVIS ROSENMAN, 525 WEST MONROE STREET, SUITE 1600, CHICAGO, IL, 60661-3693, US

(21) Appl. No.: 10/196,560

(22) Filed: Jul. 17, 2002

Related U.S. Application Data
(60) Provisional application No. 60/338,208, filed on Nov. 8, 2001.
(60) Provisional application No. 60/307,054, filed on Jul. 20, 2001.

(51) Int. Cl. A61B 6/00

(52) U.S. Cl. 600/436

(57) ABSTRACT

A system and a method for obtaining an image of a body part within a body are provided. A radiotracer including Indium-111 is administered to the body part. The system includes a first gamma ray sensor and a second gamma ray sensor, each being configured to detect prompt gamma rays emitted by Indium-111. The first gamma ray sensor is positioned external to the body, and the second gamma ray sensor is positioned either internally within the body or within a body orifice or body cavity. A relative position of the second gamma ray sensor with respect to the first gamma ray sensor may be known. The respective detections of gamma rays by the first and second gamma ray sensors may be used to determine a distribution of radioactive source material in the body part. The radiotracer may also include a positron emitter. The first and second gamma ray sensors may be configured to detect substantially coincident gamma rays emitted as a result of a positron annihilation event.

### CROSS REFERENCE TO RELATED APPLICATIONS

This application claims priority under 35 U.S.C. § 119(e) to both of the following: U.S. provisional Application Serial No. 60/307,054, entitled “Internal/External Coincident Gamma Camera System”, filed Jul. 20, 2001, and U.S. provisional Application Serial No. 60/338,208, entitled “Internal/External Coincident Gamma Camera System”, filed Nov. 8, 2001, the contents of both of which are incorporated by reference herein.

### BACKGROUND OF THE INVENTION

1.Field of the Invention

The present invention relates to an apparatus and a method for obtaining an image of a body part, and more particularly an apparatus and a method for obtaining an image using gamma radiation from a body part in a patient injected with a radioactive agent.

2. Description of the Related Art

Systems for obtaining images of body parts have been widely used by physicians, dentists and orthodontists, and other medical personnel for decades. Some of the better known conventional systems for providing images of body parts include x-ray machines, computerized axial tomography (CAT) scan machines, and magnetic resonance imaging (MRI) machines.

One objective of many physicians is to use body part imaging to provide early detection of tumors and other irregular growths that may be or become cancerous. Early detection of cancerous and precancerous growths is now recognized as a critical factor in determining whether a course of treatment will be successful. Accordingly, there is a need for accurate and precise imaging of body parts, and a corresponding need for a technique that can assist physicians with early detection of cancerous and precancerous growths in the body.

### SUMMARY OF THE INVENTION

In one aspect, the invention provides a system for obtaining an image of a body part within a body. A prompt coincidence emitting radiotracer (Indium-111, for example), is administered intravenously to the body, and accumulates preferentially in the body part. The system includes a first gamma ray sensor and a second gamma ray sensor, each being configured to detect prompt gamma rays emitted by the radiotracer. The first gamma ray sensor. is positioned external to the body, and the second gamma ray sensor is positioned either externally to the body or internally within the body. The internal sensor positioning can be achieved via a surgical incision, or via a minimally invasive route (e.g., via an endoscope), or within a naturally present body orifice or body cavity. The first gamma ray sensor may be a first gamma camera having a first parallel hole collimator that includes a first set of collimator holes having a first direction. The second gamma ray sensor may be a directional probe having a sensitive direction. A position of the directional probe with respect to a position of the first gamma camera may be known. A determination may be made as to whether a time of gamma ray detection in the directional probe and a time of gamma ray detection of energy in the first gamma camera are within a predetermined time window. The first gamma camera may detect a location of gamma ray detection events, and a first ray may be projected from the location of gamma ray detection events in parallel to the first direction. A second ray may be projected along the sensitive direction of the directional probe. An intersection of the two rays may be used to determine a distribution of radioactive source material in the body part.

The radiotracer may be a positron emitter, or a combination of positron-emitting and an emitter of prompt coincidence (e.g., Indium-111). The first and second gamma ray sensors may be further configured to detect substantially coincident gamma rays emitted as a result of a positron annihilation event. The system may also include an ultrasound camera affixed to the directional probe.

Alternatively, the second gamma ray sensor may be a compact gamma camera that includes a second collimator having a second direction. A position of the compact gamma camera with respect to a position of the first gamma camera may be known. A determination may be made as to whether a time of gamma ray detection in the compact gamma camera and a time of gamma ray detection of energy in the first gamma camera are within a predetermined time window. The first gamma camera may detect a first location of gamma ray detection events, and the compact gamma camera may detect a second location of gamma ray detection events. A first ray may be projected from the first location of gamma ray detection events in parallel to the first direction. A second ray may be projected from the second location of gamma ray detection events in parallel to the second direction. The intersection of the two rays may be used to determine a distribution of radioactive source material in the body part. The radiotracer may also include a positron emitter, and the first and second gamma ray sensors may be further configured to detect substantially coincident gamma rays emitted as a result of a positron annihilation event. The system may also include an ultrasound camera, the compact gamma camera being affixed to the ultrasound camera. The second collimator may include either a second set of parallel holes, a set of slant holes, a set of rotating slant holes, a set of parallel slits, a set of coded apertures, or a set of pinholes. Alternatively the dual sensor pair may detect radiation other than that emitted by radiotracers, and which radiation is sensed in different positions by each sensor.

The prompt coincidence-emitting radiotracer may be spin polarized prior to administration of radiotracer to the body. A magnetic field may be applied to the body part during the detection of gamma rays by the first and second gamma ray sensors. The first gamma ray sensor may be a first gamma camera having a set of collimating slits, wherein the collimating slits act as axial filters for detected gamma rays.

In another aspect, the invention provides a system for obtaining an image of a body part within a body. A prompt-coincidence emitting radiotracer including Indium-111, for example, is administered to the body part. The system includes a first gamma ray sensor and a second gamma ray sensor, both configured to detect prompt gamma rays emitted by the radiotracer. Both gamma ray sensors are positioned either internally within the body or within a body orifice or body cavity in separate locations from each other. The radiotracer may be spin polarized prior to administration to the body part. A magnetic field may be applied to the body part during the detection of gamma rays by the first and second gamma ray sensors.

In yet another aspect, the invention provides a system for obtaining an image of a body part within a body. A radiotracer including Indium-111, for example, is administered to the body part. The system includes a first gamma ray sensor and a second gamma ray sensor, both configured to detect prompt gamma rays emitted by the radiotracer. Both gamma ray sensors are positioned external to the body in separate locations from each other. The radiotracer may be spin polarized prior to administration to the body part. A magnetic field may be applied to the body part during the detection of gamma rays by the first and second gamma ray sensors.

In still another aspect, a system for obtaining an image of a body part within a body is provided. A radiotracer including Indium-111, for example, being administered to the body part. The system includes a first gamma camera and a second gamma camera. Both gamma cameras are configured to detect quasi-coincident gamma rays emitted by the radiotracer. Both gamma cameras are positioned external to the body, and both gamma cameras are directed toward a source volume. The system further includes a first one-dimensional collimator affixed to the first gamma camera and a second one-dimensional collimator affixed to the second gamma camera. An angle of orientation of the second one-dimensional collimator with respect to the first one-dimensional collimator can be varied.

In yet another aspect, the invention provides a method of obtaining an image of a body part in a body. The method includes the steps of administering a radiotracer having a radioactive ingredient to the body such that the radiotracer accumulates preferentially in a body part, positioning a first gamma ray sensor externally to the body, positioning a second gamma ray sensor either internally within the body or within a body orifice or body cavity, and using the first and second gamma ray sensors to detect gamma rays emitted by the radioactive ingredient. The radioactive ingredient may include Indium-111. The method may also include the steps of using the first gamma ray sensor to collimate a gamma ray detected by the first gamma ray sensor in a first direction, using the second gamma ray sensor to collimate a gamma ray detected by the second gamma ray sensor in a second direction, ensuring that a time of detection of the gamma ray detected by the first gamma ray sensor and a time of detection of the gamma ray detected by the second gamma ray sensor are within a predetermined time window, projecting a first ray from the first gamma ray sensor in the first direction, projecting a second ray from the second gamma ray sensor in the second direction, and determining a distribution of the radioactive ingredient within the body part on the basis of an intersection of the first and second rays.

The radioactive ingredient may include a positron emitting ingredient. The method may also include the step of using the first and second gamma ray sensors to detect substantially coincident gamma rays emitted as a result of a positron annihilation event. The method may also include the step of spin polarizing the radiotracer prior to the step of administering the radiotracer. The method may also include the step of applying a magnetic field to the body part during execution of the step of using the first and second gamma ray sensors to detect gamma rays.

In yet another aspect, the invention provides a method of obtaining an image of a body part in a body. The method includes the steps of administering a radiotracer having a radioactive ingredient to the body, such that the radiotracer accumulates preferentially in a body part, positioning a first gamma ray sensor externally to the body, positioning a second gamma ray sensor externally to the body at a separate location, and using the first and second gamma ray sensors to detect gamma rays emitted by the radioactive ingredient.

In still another aspect, the invention provides a method of obtaining an image of a body part in a body. The method includes the steps of administering a radiotracer having a radioactive ingredient to the body such that the radioactive ingredient accumulates preferentially within a body part, positioning a first gamma ray sensor either internally within the body or within a body orifice or body cavity, positioning a second gamma ray sensor either internally within the body or within a body orifice or body cavity at a separate location, and using the first and second gamma ray sensors to detect gamma rays emitted by the radioactive ingredient.

In yet another aspect of the invention, a method of obtaining an image of a body part in a body is provided. The method includes the steps of administering a radiotracer having Indium-111 to the body part, positioning a first gamma camera externally to the body and directed toward the body part, and positioning a second gamma camera externally to the body at a separate location and directed toward the body part. A first one-dimensional collimator is affixed to the first gamma camera and a second one-dimensional collimator is affixed to the second gamma camera. The method also includes the steps of varying an angle of orientation of the first one-dimensional collimator with respect to the second one-dimensional collimator, using the first and second gamma cameras to detect quasi-coincident gamma rays emitted by the Indium-111, and using the variation of angle of orientation to determine a distribution of the Indium-111 within the body part.

In yet another aspect of the invention, a method of obtaining an image of a body part in a body is provided. The method includes the steps of administering a radiating or fluorescing substance to the body which accumulates preferentially part, positioning a first camera which is sensitive to the radiation externally to the body and directed toward the body part, and positioning a second camera which is sensitive to the radiation externally to the body at a separate location and directed toward the body part, or internally within the body and directed toward the body part. The two cameras may be preferentially sensitive to different qualities of the radiation, for example one of the cameras may be more sensitive to a certain polarization of the radiation than the other. This preferential property could be used by a reconstruction algorithm implemented in a computer to determine the distribution of the radiating or fluorescing substance in the body part.

### BRIEF DESCRIPTION OF THE DRAWINGS

FIG. 1 shows a pictorial representation of a preferred embodiment of a two-component system for obtaining a image of a body part using gamma radiation detection of Indium-111.

FIG. 2 shows a pictorial representation of an alternative embodiment of a two-component system for obtaining a image of a body part using gamma radiation detection of Indium-111.

FIG. 3 shows a pictorial representation of an embodiment of a two-component system for obtaining a image of a body part using gamma radiation detection of positron emitters.

FIG. 4 shows a pictorial representation of an embodiment of a two-component system for obtaining a image of a body part using gamma radiation detection of both Indium-111 and positron emitters.

FIG. 5 shows an illustration of the correlation phenomenon between the angles of emission of cascade gamma rays emitted by Indium-111 in the presence of an external magnetic field.

FIG. 6 shows a graphical representation of the difference in coincident signal levels between two exemplary relative detector positions in the presence of an external magnetic field.

FIG. 7 shows a pictorial representation of an alternative embodiment of a two-component system for obtaining a image of a body part using gamma radiation detection of Indium-111, where one of the components is a set of collimating slits.

FIG. 8 shows a pictorial representation of an alternative embodiment of a two-component system for obtaining a image of a body part using gamma radiation detection of Indium-111, where both components are one-dimensional collimators.

### DETAILED DESCRIPTION OF THE INVENTION

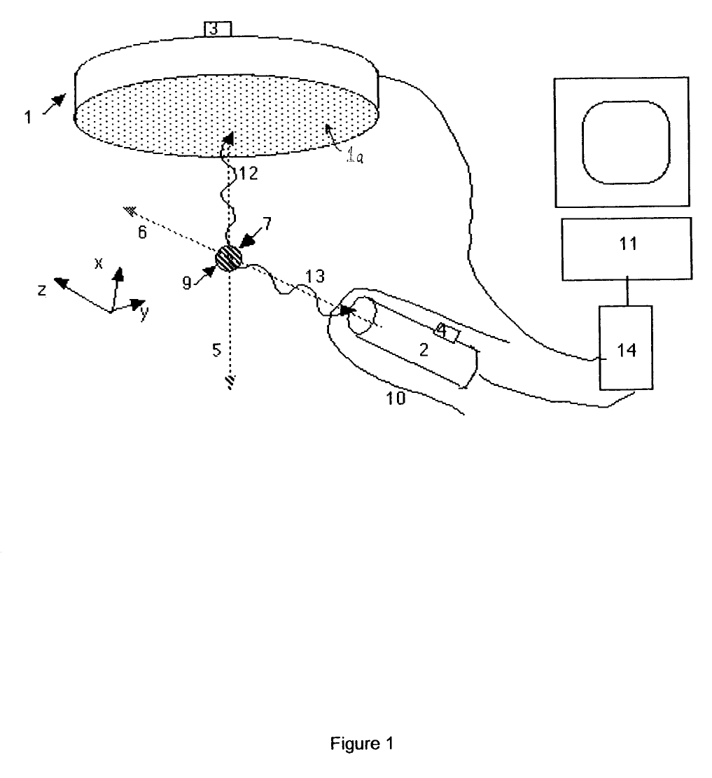

Briefly, and with reference to FIG. 1, the system according to the present invention preferably comprises two or more components sensitive to gamma radiation from a distributed source of radioactivity, such as a body part in a patient injected with radiotracer. The components' positions are transmitted to a computer for purposes of calculating the distribution of the radioactivity. Alternatively, the positions can be entered by hand, or sensed by a sensor that transmits information to the computer, or one or more of the components can travel within a known trajectory that is known to the computer so that the positions can be calculated by the computer.

In the preferred embodiment, shown in FIG. 1, a patient is injected intravenously with a radiotracer containing Indium-111. In an alternative embodiment, the radiotracer can be administered to the patient via another route. In another alternative embodiment, the radiotracer can be different from Indium-111, for example, a positron emitter can be used, in which case true coincidences are detected instead of prompt coincident events. We use the term prompt coincident events to describe radiations that are emitted within a short time interval, as opposed to coincident events as are emitted by positron emitters, in which the time interval between emissions is so short as to be essentially unmeasurable with modern electronic equipment. In this document, coincident rays from positron emissions are sometimes referred to as “simultaneous coincidences”.

An imaging system is used, which in the preferred embodiment contains two components 1 and 2, although more than two can be used as well. One component is a parallel hole gamma camera 1 that is positioned outside the body, and the other component is a directional gamma probe 2 that is positioned in a body orifice 10. A source of activity 9 has prompt gamma rays such as are emitted by Indium-111. In an alternative embodiment, both of the components can be placed inside of the body, or both can be placed outside of the body.

In this description, the terms “prompt gamma rays” and “prompt coincidences” are applied synonymously to the two gamma rays that are emitted from Indium-111 within a short time window (i.e., typically less than 90 nanoseconds), and whose angular deviations from one another are not necessarily correlated for the purposes of the preferred embodiment. In an alternative embodiment, it is possible to use the correlation between the emitted prompt gamma rays to derive physiological information about the state of the nuclide at the time of gamma-ray emission. In another alternative embodiment that involves using a positron emitter for radiotracers, it is possible to use the approximately 180-degree deviation between the emitted coincident rays to derive additional information about the position of the nuclide. When the two rays are detected that are 180 degrees opposed to one another, a line of response can be drawn between the detected events in order to perform a backprojection or reconstruction.

In the preferred embodiment, the positions of the two components 1 and 2 with respect to one another is known via position sensors 3 and 4, respectively. Preferably, the position sensors comprise electromagnetic loop sensors that are arrayed in three perpendicular planes, as in the Polhemus Tracker System. In an alternative embodiment, one of the components can be fixed and a sensor can be placed on the other component. Alternatively, one or both components can traverse a prescribed orbit at a prescribed speed or speed profile so that the sensors 3 and 4 are unnecessary. One gamma ray is emitted by an atom of Indium-111 (see item 9) that concentrates in a body part of the patient. The gamma ray which strikes the parallel hole gamma camera component 1 is localized in the x-y direction by the parallel hole collimator 1a on the gamma camera. This first localization thus defines a line 5 upon which the source must be located. When a second gamma ray that is promptly emitted following the first gamma ray is detected by the directional gamma probe component 2, a ray 6 can be projected along the direction that the aperture of gamma probe 2 is pointing (by using the position sensor 4 and rigid body mechanics to calculate the appropriate rotation and transformation matrices needed for this projection) and which intersects ray 5 at point 7. Please note that although in this figure, the direction that the probe is pointing in is the same as the long axis of the probe, this need not be the case. The long axis of the probe can have an arbitrary relationship with the direction of the aperture of the probe. Point 7 is the same as the location of the source 9. The determination of coincidence is made by a data acquisition system 14 and computer 11, one or both of which are connected to the probe, the camera, and if necessary, the position sensor or sensors 3 and 4. The data acquisition system and computer incorporate coincidence gating circuitry and reconstruction and backprojection algorithms, as described in U.S. Pat. No. 5,252,830 and U.S. Pat. application Ser. No. 09/833,110, the contents of which are incorporated herein by reference. A three-dimensional array of such intersections 7 creates a three-dimensional map of the distribution of the radioactive source in the volume beneath the parallel hole gamma camera. The internal directional probe 2 can be affixed to an ultrasound camera, or can fit within a fixture to which an ultrasound camera can be fitted so that the ultrasound camera and the probe are co-registered.

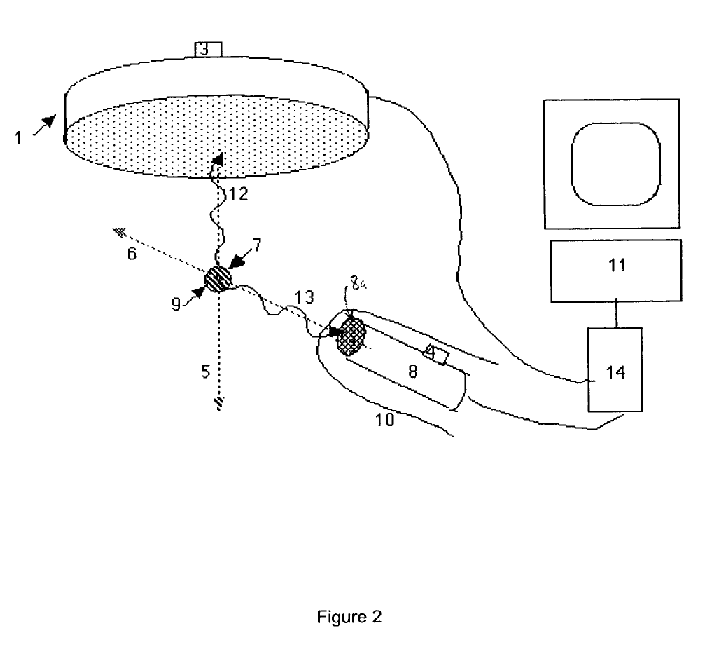

Referring to FIG. 2, in an alternative embodiment, the directional gamma probe 2 is replaced with a small gamma camera 8, which may contain a collimator 8a using holes that are parallel or otherwise placed (e.g., rotating slant hole, slant hole, coded aperture, parallel slits, pinhole).

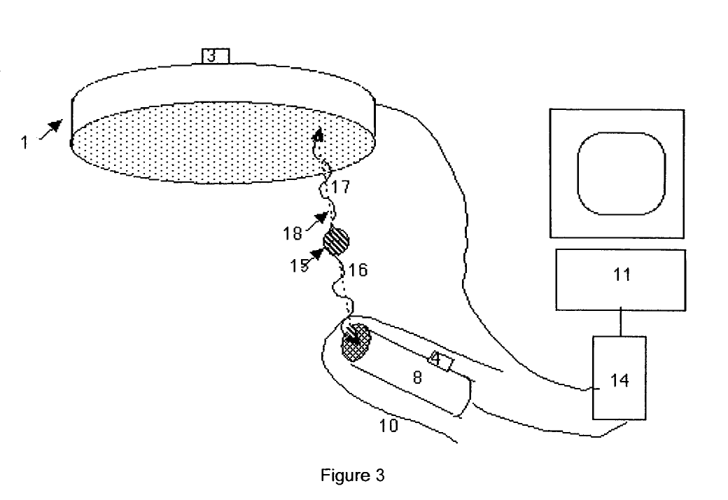

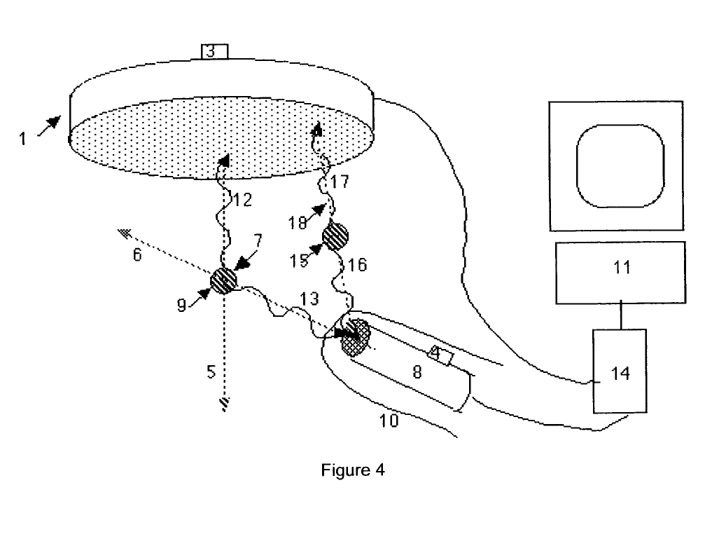

Referring to FIG. 3, in another alternative embodiment, the imaging system components 1 and 2, or alternatively 1 and 8 described above, can be used to detect positron emitters 15 that emit coincident gamma rays 16 and 17 instead of prompt gamma rays, as are emitted by Indium-111. In this case, a line of coincidence 18 is drawn between the imaging system components. Referring to FIG. 4, in another alternative embodiment, the imaging system components 1 and 2, or alternatively 1 and 8 described above, can be used to simultaneously detect coincident and prompt gamma ray emitting radioactive source by selecting energy ranges appropriate for each gamma emitter (e.g., 511 keV for the positron emitters, and lower energies for Indium-111).

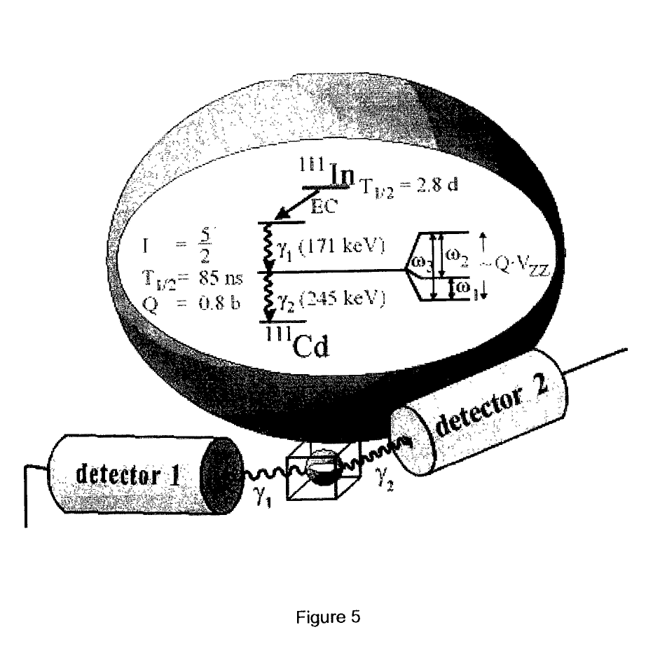

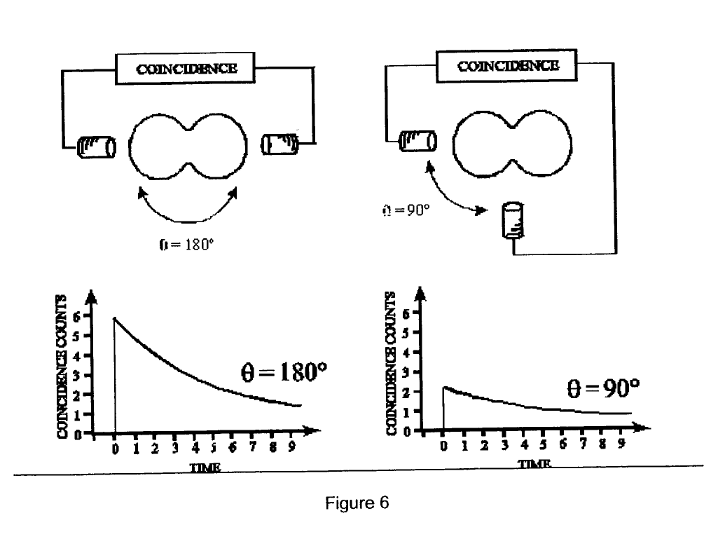

Referring to FIG. 5, it is known that in the application of an external magnetic field, the angles of emission of the cascade gamma rays emitted by Indium-111 are correlated. For example, referring also to FIG. 6, in a high magnetic field, detectors placed on both sides of a decaying atom will have higher coincident signals than if they are placed at right angles to one another. The invention can take advantage of this phenomenon by placing detectors on both sides of the body part of interest as described above and also performing one or both of the following: 1) applying a magnetic field to the Indium-111 before administration to the patient to spin-polarize the sample, so that the gamma rays are correlated until there is time for the spin polarization to wear off; and/or 2) placing the patient in a magnetic field. Note that if the angles are 180 degrees apart, it is possible to dispense with or reduce the need for collimators on the gamma camera components. The scientific phenomena illustrated in FIGS. 5 and 6 are more fully described in the following publications, both of which are incorporated herein by reference: 1) “Indium-Hg vacancy interactions I Hg 1-x, Cdx, Te measured by perturbed angular correlation”, W. C. Hughes et al., Applied Physics Lett. 59(8), Aug. 19, 1991, available on the Internet at http://csm.jmu.edu/physics/hughes/APL_{—}59_{—}938_{—}1991%20.pdf. 2) Internet web site address http://216.239.51.100/search?q=cache:scvdsO0-314C:www.jlab.org/div_dept/detector/docs/detector.ps+coincidence+indium+180&h1=en&ie=UTF-8.

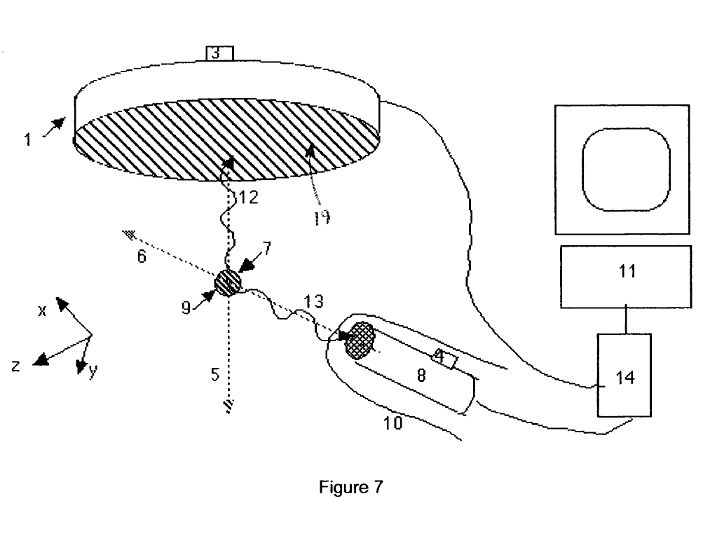

Referring to FIG. 7, in an alternative embodiment, the parallel hole collimator on camera 1 shown in FIG. 2 can be replaced with a set of collimating slits 19 (i.e., axial filters, as have been used in Marconi brand hybrid coincident gamma cameras), which are more efficient than a parallel hole collimator and yet serve to provide z-localization, thereby improving the quality of the three-dimensional map. Note that this would work if the internal gamma ray sensor was a two-dimensional imager (i.e., a camera), or was a non-imaging directional probe such as that shown in FIG. 1, or had an axial filter collimator.

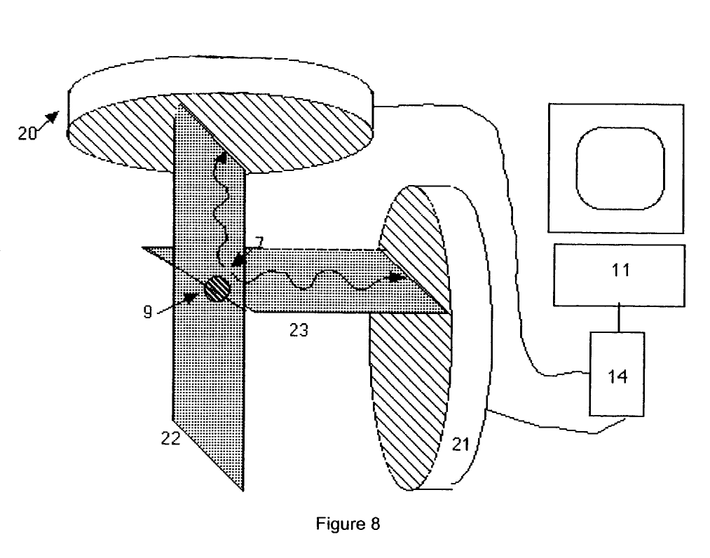

Referring to FIG. 8, in another alternative embodiment, the components comprising the system include two gamma cameras 20 and 21 directed toward a source volume, each camera being equipped with one-dimensional collimators that are oriented at various angles, including perpendicularly (and whose collimator may rotate), to one another, and when gamma rays arrive at both gamma cameras within a coincident time window, the intersections of the planes 22 and 23 backprojected from both gamma camera heads defines the location of the source 9.

In another alternative embodiment, attenuation correction can be implemented by placing a point source or multiple sources on or affixed to one or more detector components.

In another alternative embodiment, position sensing may also be implemented by affixing one or more radioactive sources in a known configuration to one or more detector components, preferably a component that is mobile. Position sensing can be accomplished by viewing the radioactive fiducial sources from various perspectives, as has been implemented in tomosynthesis methods used in Instrumentarium x-ray mammography cameras. The foregoing applies for coincident imaging with positron emitters or coincident non-positron emitters, such as Indium-111, or non-coincident imaging with single photon emitters or with other systems with hand-held components that can detect signals.

The alternative embodiments set forth herein are by way of example and not limitation.

While the present invention has been described with respect to what is presently considered to be the preferred embodiment, it is to be understood that the invention is not limited to the disclosed embodiments. To the contrary, the invention is intended to cover various modifications and equivalent arrangements included within the spirit and scope of the appended claims. For example, although the use of Indium-111 and/or a positron emitter within the radiotracer is preferred, it is to be understood that the invention is applicable to any radiotracer that emits gamma rays and that can be administered safely to a patient. As another example, although a parallel hole collimator is described as being part of the preferred embodiment, other types of collimators, including those that use parallel slits, pinholes, slant holes, rotating slant holes, or coded apertures may also be used. The scope of the following claims is to be accorded the broadest interpretation so as to encompass all such modifications and equivalent structures and functions.

What is claimed is:

1. A system for obtaining an image of a body part within a body, a radiotracer including Indium-111 being administered intravenously to the body such that the radiotracer accumulates preferentially in the body part, and the system comprising:
a first gamma ray sensor configured to detect prompt gamma rays emitted by Indium-111, the first gamma ray sensor being positioned external to the body, and
a second gamma ray sensor configured to detect prompt gamma rays emitted by Indium-111, the second gamma ray sensor being positioned either internally within the body or within a body orifice or body cavity.

2. The system of claim 1, wherein the first gamma ray sensor comprises a first gamma camera having a first parallel hole collimator, the first parallel hole collimator including a first set of collimator holes having a first direction.

3. The system of claim 2, further comprising a computer, the computer being in communication with the first and second gamma ray sensors, and a coincident gate electronically coupled to the computer, wherein
the second gamma ray sensor comprises a directional probe, the directional probe having a sensitive direction, and wherein
a position of the directional probe with respect to a position of the first gamma camera is known, and wherein
a determination is made by the coincident gate and the computer as to whether a time of gamma ray detection in the directional probe and a time of gamma ray detection of energy in the first gamma camera are within a predetermined time window.

4. The system of claim 3, wherein
the first gamma camera detects a location of gamma ray detection events, and wherein a first ray is projected from the location of gamma ray detection events, the first ray being parallel to the first direction, and wherein
a second ray is projected along the sensitive direction of the directional probe, and wherein
the computer is configured to execute a reconstruction or backprojection algorithm using an intersection of the two rays to determine a distribution of radioactive source material in the body part.

5. The system of claim 4, wherein the radiotracer further includes a positron emitter, and the first and second gamma ray sensors are further configured to detect substantially coincident gamma rays emitted as a result of a positron annihilation event.

6. The system of claim 4, further comprising an ultrasound camera, the directional probe being affixed to the ultrasound camera.

7. The system of claim 2, further comprising a computer in communication with the first and second gamma ray sensors, wherein
the second gamma ray sensor comprises a second gamma camera having a second collimator, the second collimator having a second direction, and wherein
a position of the second gamma camera with respect to a position of the first gamma camera is known, and wherein
the computer executes a reconstruction or backprojection algorithm to make a determination as to whether a time of gamma ray detection in the second gamma camera and a time of gamma ray detection of energy in the first gamma camera are within a predetermined time window.

8. The system of claim 7, wherein
the first gamma camera detects a first location of gamma ray detection events, and
the second gamma camera detects a second location of gamma ray detection events, and
a first ray is projected from the first location of gamma ray detection events, the first ray being parallel to the first direction, and wherein
a second ray is projected from the second location of gamma ray detection events, the second ray being parallel to the second direction, and wherein
the intersection of the two rays is used to determine, by the computer executing the reconstruction or backprojection algorithm, a distribution of radioactive source material in the body part.

9. The system of claim 8, wherein the radiotracer further a positron emitter, and the first and second gamma ray sensors are further configured to detect substantially coincident gamma rays emitted as a result of a positron annihilation event.

10. The system of claim 8, further comprising an ultrasound camera, the compact gamma camera being affixed to the ultrasound camera.

11. The system of claim 7, wherein the second collimator includes a second set of parallel holes.

12. The system of claim 7, wherein the second collimator includes a set of slant holes.

13. The system of claim 7, wherein the second collimator includes a set of rotating slant holes.

14. The system of claim 7, wherein the second collimator includes a set of parallel slits.

15. The system of claim 7, wherein the second collimator includes a set of coded apertures.

16. The system of claim 7, wherein the second collimator includes a set of pinholes.

17. The system of claim 1, wherein the radiotracer further includes a positron emitter, and the first and second gamma ray sensors are further configured to detect substantially coincident gamma rays emitted by positron emission.

18. The system of claim 1, wherein the radiotracer is spin polarized prior to administration of the radiotracer to the body part.

19. The system of claim 1, wherein a magnetic field is applied to the body part during the detection of gamma rays by the first and second gamma ray sensors.

20. The system of claim 1, wherein the radiotracer is spin polarized prior to administration of the radiotracer to the body part, and wherein a magnetic field is applied to the body part during the detection of gamma rays by the first and second gamma ray sensors.

21. The system of claim 1, wherein the first gamma ray sensor comprises a first gamma camera having a set of collimating slits, wherein the collimating slits act as axial filters for detected gamma rays.

22. A system for obtaining an image of a body part within a body, a radiotracer including Indium-111 being administered intravenously to the body such that the radiotracer accumulates preferentially in the body part, and the system comprising:
a first gamma ray sensor and a second gamma ray sensor, both gamma ray sensors being configured to detect prompt gamma rays emitted by Indium-111, and both gamma ray sensors being positioned either internally within the body or within a body orifice or body cavity,
wherein the second gamma ray sensor is positioned at a separate location from the first gamma ray sensor.

23. The system of claim 22, wherein the radiotracer is spin polarized prior to administration of the radiotracer to the body part.

24. The system of claim 22, wherein a magnetic field is applied to the body part during the detection of gamma rays by the first and second gamma ray sensors.

25. The system of claim 22, wherein the radiotracer is spin polarized prior to administration of the radiotracer to the body part, and wherein a magnetic field is applied to the body part during the detection of gamma rays by the first and second gamma ray sensors.

26. A system for obtaining an image of a body part within a body, a radiotracer including Indium-111 being administered intravenously to the body such that the radiotracer accumulates preferentially in the body part, and the system comprising:
a first gamma ray sensor and a second gamma ray sensor, both gamma ray sensors being configured to detect prompt gamma rays emitted by Indium-111, and both gamma ray sensors being positioned external to the body,
wherein the second gamma ray sensor is positioned at a separate location from the first gamma ray sensor.

27. The system of claim 26, wherein the radiotracer is spin polarized prior to administration of the radiotracer to the body part.

28. The system of claim 26, wherein a magnetic field is applied to the body part during the detection of gamma rays by the first and second gamma ray sensors.

29. The system of claim 26, wherein the radiotracer is spin polarized prior to administration of the radiotracer to the body part, and wherein a magnetic field is applied to the body part during the detection of gamma rays by the first and second gamma ray sensors.

30. A system for obtaining an image of a body part within a body, a radiotracer including Indium-111 being administered to the body part, and the system comprising:
a first gamma camera and a second gamma camera, both gamma cameras being configured to detect quasi-coincident gamma rays emitted by Indium-111, and both gamma cameras being positioned external to the body, and both gamma cameras being directed toward a source volume;
a first one-dimensional collimator affixed to the first gamma camera; and
a second one-dimensional collimator affixed to the second gamma camera, wherein
said first and second collimators being configured such that an angle of orientation of the second one-dimensional collimator with respect to the first one-dimensional collimator can be varied.

31. A system for obtaining an image of a body part within a body, a radiotracer including a positron emitter being administered intravenously to the body such that the radiotracer accumulates preferentially in the body part, and the system comprising:
a first gamma ray sensor configured to detect substantially coincident gamma rays emitted by the positron emitter, the first gamma ray sensor being positioned external to the body, and
a second gamma ray sensor configured to detect substantially coincident gamma rays emitted by the positron emitter, the second gamma ray sensor being positioned either internally within the body or within a body orifice or body cavity.

32. A system for obtaining an image of a body part within a body, a radiotracer including a positron emitter being administered intravenously to the body such that the radiotracer accumulates preferentially in the body part, and the system comprising:
a first gamma ray sensor and a second gamma ray sensor, both gamma ray sensors being configured to detect substantially coincident gamma rays emitted by the positron emitter, and both gamma ray sensors being positioned either internally within the body or within a body orifice or body cavity,
wherein the second gamma ray sensor is positioned at a separate location from the first gamma ray sensor.

33. A system for obtaining an image of a body part within a body, a radiotracer including a positron emitter being administered intravenously to the body such that the radiotracer accumulates preferentially in the body part, and the system comprising:
a first gamma ray sensor and a second gamma ray sensor, both gamma ray sensors being configured to detect substantially coincident gamma rays emitted by the positron emitter, and both gamma ray sensors being positioned external to the body,
wherein the second gamma ray sensor is positioned at a separate location from the first gamma ray sensor.

34. An apparatus for determining a distribution of radioactive source material in a body part to which a radiotracer is administered, the apparatus comprising:
a first means for sensing gamma rays positioned external to the body;
a means for determining a first direction of gamma rays sensed by the first means for sensing;
a second means for sensing gamma rays positioned either internally within the body or within a body orifice or body cavity; and
a means for determining a second direction of gamma rays sensed by the second means for sensing.

35. The apparatus of claim 34, wherein the radioactive source material includes Indium-111, and the first and second means for sensing are configured to sense prompt gamma rays, and the apparatus further comprises a means for determining whether a time of sensing by the first means for sensing is within a predetermined time interval of a time of sensing by the second means for sensing.

36. The apparatus of claim 34, wherein the radioactive source material includes a positron emitter, and the first and second means for sensing are configured to sense substantially coincident gamma rays emitted by positron emission.

37. The apparatus of claim 34, wherein the radioactive source material includes Indium-111 and a positron emitter, and the first and second means for sensing are configured to sense prompt gamma rays emitted by Indium-111 and substantially coincident gamma rays emitted by positron emission, and the apparatus further comprises a means for determining whether a time of sensing prompt gamma rays by the first means for sensing is within a predetermined time interval of a time of sensing prompt gamma rays by the second means for sensing.

38. The apparatus of claim 34, wherein the radiotracer is spin polarized prior to administration to the body part.

39. The apparatus of claim 34, further comprising a means for applying a magnetic field to the body part.

40. The apparatus of claim 39, wherein the radiotracer is spin polarized prior to administration to the body part.

41. An apparatus for determining a distribution of radioactive source material in a body part to which a radiotracer is administered, the apparatus comprising:
a first means for sensing gamma rays positioned either internally within the body or within a body orifice or body cavity;
a means for determining a first direction of gamma rays sensed by the first means for sensing;
a second means for sensing gamma rays positioned either internally within the body or within a body orifice or body cavity at a separate location from the first means for sensing; and
a means for determining a second direction of gamma rays sensed by the second means for sensing.

42. An apparatus for determining a distribution of radioactive source material in a body part to which a radiotracer is administered, the apparatus comprising:
a first means for sensing gamma rays positioned external to the body;
a means for determining a first direction of gamma rays sensed by the first means for sensing;
a second means for sensing gamma rays positioned external to the body at a separate location from the first means for sensing; and
a means for determining a second direction of gamma rays sensed by the second means for sensing.

43. The apparatus of claim 42, wherein the radioactive source material includes Indium-111, and the first means for sensing includes a first one-dimensional means for collimating a sensed quasi-coincident gamma ray emitted by Indium-111, and the second means for sensing includes a second one-dimensional means for collimating a sensed quasi-coincident gamma ray emitted by Indium-111, and the apparatus further comprises a means for varying an angle of orientation of the second one-dimensional means for collimating with respect to the first one-dimensional means for collimating.

44. A method of obtaining an image of a body part in a body, comprising the steps of:
administering a radiotracer having a radioactive ingredient to the body part;
positioning a first gamma ray sensor externally to the body;
positioning a second gamma ray sensor either internally within the body or within a body orifice or body cavity; and
using the first and second gamma ray sensors to detect gamma rays emitted by the radioactive ingredient.

45. The method of claim 44, wherein the radioactive ingredient is Indium-111.

46. The method of claim 45, further comprising the steps of:
using the first gamma ray sensor to collimate a gamma ray detected by the first gamma ray sensor in a first direction;
using the second gamma ray sensor to collimate a gamma ray detected by the second gamma ray sensor in a second direction;
ensuring that a time of detection of the gamma ray detected by the first gamma ray sensor and a time of detection of the gamma ray detected by the second gamma ray sensor are within a predetermined time window;
projecting a first ray from the first gamma ray sensor in the first direction;
projecting a second ray from the second gamma ray sensor in the second direction; and
determining a distribution of the radioactive ingredient within the body part on the basis of an intersection of the first and second rays.

47. The method of claim 44, wherein the radioactive ingredient is a positron emitting ingredient.

48. The method of claim 47, further comprising the step of using the first and second gamma ray sensors to detect substantially coincident gamma rays emitted as a result of a positron annihilation event.

49. The method of claim 44, wherein the radioactive ingredient includes Indium-111 and a positron emitting ingredient.

50. The method of claim 49, further comprising the steps of:
using the first gamma ray sensor to collimate a gamma ray detected by the first gamma ray sensor in a first direction;
using the second gamma ray sensor to collimate a gamma ray detected by the second gamma ray sensor in a second direction;
ensuring that a time of detection of the gamma ray detected by the first gamma ray sensor and a time of detection of the gamma ray detected by the second gamma ray sensor are within a predetermined time window;
projecting a first ray from the first gamma ray sensor in the first direction;
projecting a second ray from the second gamma ray sensor in the second direction;
using the first and second gamma ray sensors to detect substantially coincident gamma rays emitted as a result of a positron annihilation event; and
determining a distribution of the radioactive ingredient within the body part on the basis of an intersection of the first and second rays and on the basis of the detected substantially coincident gamma rays.

51. The method of claim 44, further comprising the step of spin polarizing the radiotracer prior to the step of administering the radiotracer.

52. The method of claim 44, further comprising the step of applying a magnetic field to the body part during execution of the step of using the first and second gamma ray sensors to detect gamma rays.

53. The method of claim 52, further comprising the step of spin polarizing the radiotracer prior to the step of administering the radiotracer.

54. A method of obtaining an image of a body part in a body, comprising the steps of:
administering a radiotracer having a radioactive ingredient to the body part;
positioning a first gamma ray sensor externally to the body;
positioning a second gamma ray sensor externally to the body at a separate location from the first gamma ray sensor; and
using the first and second gamma ray sensors to detect gamma rays emitted by the radioactive ingredient.

55. A method of obtaining an image of a body part in a body, comprising the steps of:
administering a radiotracer having a radioactive ingredient to the body part;
positioning a first gamma ray sensor either internally within the body or within a body orifice or body cavity;
positioning a second gamma ray sensor either internally within the body or within a body orifice or body cavity at a separate location from the first gamma ray sensor; and
using the first and second gamma ray sensors to detect gamma rays emitted by the radioactive ingredient.

56. A method of obtaining an image of a body part in a body, comprising the steps of:
administering a radiotracer having Indium-111 to the body part;
positioning a first gamma camera externally to the body and directed toward the body part, wherein a first one-dimensional collimator is affixed to the first gamma camera;
positioning a second gamma camera externally to the body, at a separate location from the first gamma ray sensor and directed toward the body part, wherein a second one-dimensional collimator is affixed to the second gamma camera;
varying an angle of orientation of the first one-dimensional collimator with respect to the second one-dimensional collimator;
using the first and second gamma cameras to detect quasi-coincident gamma rays emitted by the Indium-111; and using the variation of angle of orientation to determine a distribution of the Indium-111 within the body part.
